(19) United States
(12) Patent Application Publication
(10) Pub. No.: US 2011/0237676 A1
(43) Pub. Date: Sep. 29, 2011

(54) SPERMICIDAL AND MICROBICIDAL COMPOSITIONS

(75) Inventors: Barry Bean, Bethlehem, PA (US); Robert A Flowers, Nazareth, PA (US); Jennifer J Venditti, Bethlehem, PA (US); Rajni Singh, Bethlehem, PA (US)

(73) Assignee: LEHIGH UNIVERSITY, Bethlehem, PA (US)

(21) Appl. No.: 12/674,342

(22) PCT Filed: Aug. 20, 2008
(86) PCT No.: PCT/US08/73730

§ 371 Date: May 20, 2011

Related U.S. Application Data
(60) Provisional application No. 60/956,960, filed on Aug. 21, 2007.

(51) Int. Cl. A61K 31/185; C12N 5/00; C07C 43/12; A61K 31/08; A61P 35/04; A61P 31/12; A61P 31/22; C07C 309/04; A61P 15/16

(52) U.S. Cl. 514/578; 435/375; 568/615; 514/723; 562/110

(57) ABSTRACT

Provided herein are new spermicidal compositions that include one or more fluorous surfactants, e.g., non-ionic fluorous surfactants, anionic fluorous surfactants, cationic fluorous surfactants, and combinations thereof. Also provided are methods of preventing fertilization of an ovum, which include a step of contacting at least one spermatozoon with one or more fluorous surfactants. Additionally described herein are methods of preventing infection, which include a step of contacting at least one infectious pathogenic microorganism with one or more fluorous surfactants. Numerous fluorine-containing surfactants are demonstrated to be efficacious as human spermicides.

### CROSS REFERENCE TO RELATED APPLICATIONS

This application claims the priority of co-pending U.S. provisional patent application No. 60/956,960, filed Aug. 21, 2007, the entire contents of which are incorporated herein by reference.

### FIELD

This application describes novel spermicidal and microbicidal compositions that contain fluorous surfactants and methods of use thereof.

### BACKGROUND

Spermicides exert an anti-fertility effect upon spermatozoa as it passes through the female genital tract. To be an effective contraceptive agent, a compound should meet certain requirements. It should act rapidly and efficiently to kill or immobilize sperm on contact or otherwise render sperm incapable of fertilization. It also should be suitable for administration; that is, it should not be unnecessarily irritating to the vaginal and penile mucosa. Furthermore, it should be free of long-term toxicity, and it should be systemically non-toxic.

At present, commercially available spermicidal contraceptives have detergent ingredients that disrupt cell membranes, principally because of their affinity to the membrane lipids. These include the surfactants nonoxynol-9 (“N-9”), menfegol, octoxynol-9 (“O-9”), and Triton-X®, among others (e.g., sodium dodecyl sulfate (“SDS”) and cetyl trimethylammonium bromide (“CTAB”)). [TRITON-X® is a registered trademark of Union Carbide Chemicals & Plastics Technology Corp. of Indianapolis, Ind.]. N-9 is the most commonly used spermicidal contraceptive in the United States.

Unfortunately, these detergents, especially N-9, have been shown to damage the cell lining of the vagina and cervix, thereby increasing the risk of STD transmission. Despite its widespread use, N-9 is associated with damage to cells other than sperm, and it is known to cause small tissue lesions. In fact, the use of spermicides containing N-9 has been linked to a greater risk of HIV transmission. It has therefore been proposed that contraceptives containing N-9 include a warning label that advises the user of the risks associated with N-9, such as advice to consumers that the use of vaginal contraceptives containing N-9 can increase vaginal irritation, which may actually increase the possibility of transmitting the AIDS virus and other STDs from infected partners.

There exists a continuing and unmet need for improved spermicidal compositions that are effective in preventing fertilization but do not suffer from one or more of the deficiencies of currently available products.

### SUMMARY

Provided herein are new spermicidal compositions that include one or more fluorous surfactants, e.g., non-ionic fluorous surfactants, anionic fluorous surfactants, cationic fluorous surfactants, and combinations thereof. Also provided are methods of preventing fertilization of an ovum, which include a step of contacting at least one spermatozoon with one or more fluorous surfactants. Additionally described herein are methods of preventing infection, which include a step of contacting at least one infectious pathogenic microorganism with one or more fluorous surfactants. Numerous fluorine-containing surfactants are demonstrated to be efficacious as human spermicides. Additionally, methods of use for those surfactants include application to spermatozoa for degrading membranes useful in selective reduction of viable spermatozoa and for selective extraction of cellular materials from spermatozoa.

As described in more detail herein below, whole semen was treated with varying concentrations of fluorous surfactants. Motility, propidium iodide staining, and FITC-PSA staining of sperm cells were evaluated microscopically. Results from these experiments indicated that fluorous surfactants are effective at killing sperm cells. For example, within five minutes of treatment at the concentrations used, nearly all sperm cells were non-motile and the majority of them were stained with propidium iodide, which is a DNA intercalating agent that indicates cell necropsy (it only passes through compromised cell membranes). FITC-PSA stain, which shows the status of the acrosomal membrane, indicated that these surfactants compromise the acrosomal region of the sperm thereby preventing ovum fertilization.

Additional features may be understood by referring to the accompanying Drawings, which should be read in conjunction with the following Detailed Description and Examples.

### DESCRIPTION OF THE DRAWINGS

FIG. 1 illustrates some example surfactants used in commercially available spermicides.

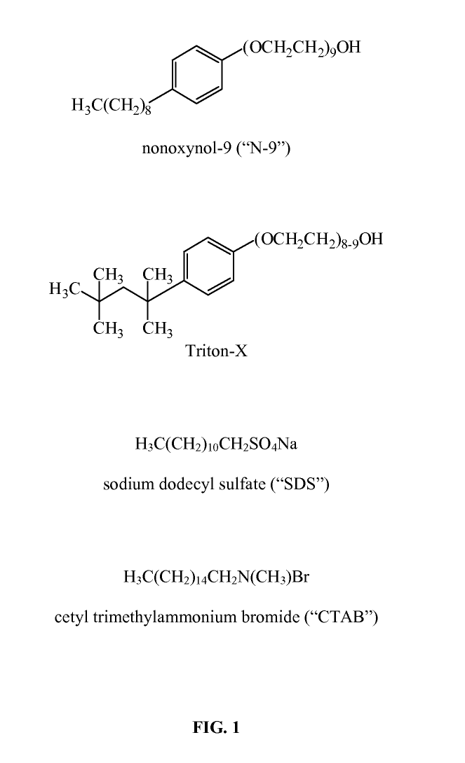

FIG. 2 illustrates some fluorous surfactants in accordance with examples embodiments hereof.

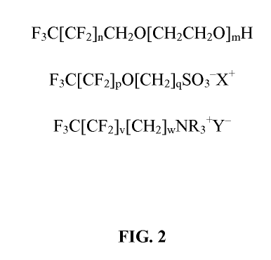

### DETAILED DESCRIPTION AND EXAMPLE EMBODIMENTS

Fluorinated compounds have several uniquely advantageous features. For example, fluorous solvents are immiscible in both aqueous and organic media, and the human body lacks enzymes to break down fluorous compounds, yet they are easy to remove from proteins. Fluorous surfactants, e.g., cationic, anionic, and nonionic surfactants, with various combinations of hydrocarbon and fluorocarbon components, interact with purified proteins differently than comparable hydrocarbon detergents. For example, fluorous surfactants have a lower critical micelle concentration than their corresponding hydrocarbon homologs.

Indeed, some fluorous compounds produce gentle association with particular proteins, resulting in little denaturation and thereby allowing for repurification and refolding of the protein to restore enzymatic function. Fluorinated surfactants are thought to have more gentle interactions with human tissues and cells. Moreover, fluorous surfactants are useful for the selective extraction of superficial components of biological membranes (e.g., from sperm), while minimizing damage to surrounding biologic structures. As further described herein, low affinity fluorous surfactants are demonstrated to disrupt sperm cell membranes, while mitigating the deleterious effects of non-fluorinated surfactants that are used in conventional spermicides.

The architecture of human sperm cells is complex, including several distinctive membrane regions with different compositions. These membranous surfaces undergo substantial changes during the life history of the sperm cell, and they serve as boundaries that partition the sperm cell into compartments and surfaces that have different functional capabilities. Most important are sperm functions that enable fertilization. Upon ejaculation, the mature sperm cell is poised to undergo a series of structural changes that constitute the essential pathway toward successful fertilization. Fluorous surfactants alter sperm membranes differentially, compromising the acrosomal, flagellar, or other sperm membranes, with consequent reduction in fertilization potential.

As described in the Examples, whole semen was treated with varying concentrations of fluorous surfactants. Motility, propidium iodide staining, and FITC-PSA staining of sperm cells were evaluated microscopically. Results from these experiments indicated that fluorous surfactants are effective at killing sperm cells. For example, within five minutes of treatment at the concentrations used, nearly all sperm cells were non-motile and the majority of them were stained with propidium iodide. Likewise, under some treatment conditions, FITC-PSA stain indicated that these surfactants compromise the acrosomal region of the sperm. In general, it was observed that exposing sperm to the fluorous surfactants resulted in a greater amount of compromised sperm. Although all fluorous surfactants were effective in immobilizing the sperm without necessarily killing them at the concentrations tested, increasing concentrations of surfactants do not linearly correlate with effectiveness. Therefore, each fluorous surfactant may have its own unique optimal concentration to accomplish a desired effect.

Accordingly, in an example embodiment hereof, a method of preventing fertilization of an ovum includes a step of contacting at least one spermatozoon (e.g., a human spermatozoon) with one or more fluorous surfactants. Typically, the one or more fluorous surfactants each include at least one fluorine atom, have a molecular weight under about 500, and are at least partially soluble in water. The one or more fluorous surfactants may include non-ionic fluorous surfactants, anionic fluorous surfactants, cationic fluorous surfactants, and combinations thereof.

Suitable non-ionic fluorous surfactants include compounds having a fluorine-substituted lower alkyl group covalently bonded to a polyoxyalkyl group (e.g., a polyoxyethylene group). As used herein, a “fluorine-substituted lower alkyl group” may be a methyl, ethyl, propyl, butyl, pentyl, hexyl, heptyl, octyl, nonyl, decyl, undecyl, dodecyl, or larger alkyl group, which may be branched or unbranched, in which at least one hydrogen atom has been substituted by a fluorine atom. For example, a non-ionic fluorous surfactant may be a compound having the chemical structure F₃C[CF₂]ₙCH₂O[CH₂CH₂O]ₘH, wherein n is an integer from 1 to 10 (e.g., n is an integer from 1 to 5), and m is an integer from 1 to 10 (e.g., m is an integer from 3 to 6).

Additional suitable anionic fluorous surfactants include compounds having a fluorine-substituted lower alkyl group covalently bonded via an oxygen atom to a lower alkyl sulfonic acid. As used herein, a “lower alkyl sulfonic acid” may be a methyl, ethyl, propyl, butyl, pentyl, hexyl, heptyl, octyl, nonyl, decyl, undecyl, dodecyl, or larger alkly group, which may be branched or unbranched, in which at least one hydrogen atom has been substituted by a sulfonate (SO₃⁻) group. For example, an anionic fluorous surfactant may be a compound having the chemical structure F₃C[CF₂]ₚO[CH₂]_{q}SO₃⁻X⁺, wherein p is an integer from 1 to 10 (e.g., p is an integer from 1 to 7), q is an integer from 1 to 10 (e.g., q is an integer from 3 to 6), and X⁺ is a physiologically acceptable cation. As used herein, a “physiologically acceptable cation” includes any cationic species, such as, for example, sodium, lithium, potassium, and tetraalkyl ammonium ions, that within sound medical judgment do not entirely inhibit the desired spermicidal or microbicidal actions of the compound and that counter balance the negative charge of the sulfonate group. The group X⁺ not intended to be limited to ions with only a +1 charge; it may include any cationic species of appropriate stoicheometry.

Examples of suitable cationic fluorous surfactants include compounds having a fluorine-substituted lower alkyl group covalently bonded to a lower alkyl ammonium ion. As used herein, a “lower alkyl ammonium ion” may be a methyl, ethyl, propyl, butyl, pentyl, hexyl, heptyl, octyl, nonyl, decyl, undecyl, dodecyl, or larger alkyl group, which may be branched or unbranched, in which at least one hydrogen atom has been substituted by a ammonium (NR₃⁺) group, wherein each “R” is independently a hydrogen or a methyl, ethyl, propyl, butyl, pentyl, hexyl, heptyl, octyl, nonyl, decyl, undecyl, dodecyl, etc. group, which may be branched or unbranched. For example, a cationic fluorous surfactant may be a compound having the chemical structure F₃C[CF₂]ᵥ[CH₂]_{w}NR₃³⁰ Y⁻, wherein v is an integer from 1 to 10 (e.g., v is an integer from 1 to 7), w is an integer from 1 to 10 (e.g., w is an integer from 3 to 6), each R is independently a hydrogen or lower alkyl group, and Y⁻ is a physiologically acceptable anion. As used herein, a “physiologically acceptable anion” includes any anionic species, such as, for example, chloride and bromide ions, that within sound medical judgment do not entirely inhibit the spermicidal or microbicidal actions of the compound and that counter balance the positive charge of the ammonium group. The group Y⁻ is not intended to be limited to ions with only a −1 charge; it may include any anionic species of appropriate stoicheometry.

Also provided herein is a novel spermicidal composition, which may include one or more fluorous surfactants compounds described herein. The spermicidal composition may also include one or more additional agents such as emulsifiers, gelling agents, stabilizers, antioxidants, osmolality (or osmolarity) adjusting agents, buffers, isotonic agents, water, glycerol, propylene glycol, liquid silicone (e.g., Silastic® silicone elastomers), liquid polyethylene glycol, lipids, lubricants, perfumes, flavoring agents, dyes, preservatives, and combinations thereof. The spermicidal composition may be in the form of a latex-compatible liquid solution, suspension, or dispersion, for example. [SILASTIC® is a registered trademark of Dow Corning Corp. of Midland, Mich.]. The composition may be lubricious, such as a personal lubricant or vaginal moisturizer. It may be provided in any suitable dosage form, such as liquid, foam, sponge, or suppository, for example.

In yet another embodiment, a method of preventing infection includes a step of contacting at least one infectious pathogenic microorganism with one or more fluorous surfactants. Infectious pathogenic microorganism include Neisseria gonorrhoeae (gonorrhoea), Chlamydia trachomatis (chlamydia), Staphylococcus aureus (staph), Treponema pallidum (syphilis), Trichomonas vaginalis (trichomoniasis), Candida albicans (yeast), Trichophyton rubrum (ringworm), Escherichia coli, hepatitis virus (including hepatitis A, B, C, D, E, F, and G), cytomegalovirus, herpes simplex virus (including HSV-1 and HSV-2), human immunodeficiency virus (including HIV-1 and HIV-2), human papillomavirus (including types 16, 18, 31, 33, 35, 39, 45, 51, 52, 56, 58, 59, and 68), and molluscum contagiosum virus (including types 1, 2, 3 and 4). The step of contacting may be in vivo or in vitro, and may be for medical or diagnostic purposes, for example.

### EXAMPLES

### Example 1

### Evaluation of Fluorous Surfactants as Spermicides

The general experimental protocol was as follows: Whole semen, obtained from human subjects after informed consent, was treated with various concentrations of fluorous surfactants. Samples were incubated for 5-10 min at 37° C. and 5% CO₂. Semen samples were diluted in an isotonic buffered human sperm medium containing glucose. Sperm motility was scored as “% motility” using differential interference microscopy (“DIC”). Cell necropsy was determined by propidium iodide (“PI”) staining, which is known to stain the DNA of dead cells, and “% death” was scored using epifluorescence microscopy (stained and unstained sperm cells were counted to obtain a live/dead ratio). Acrosome integrity was determined with epifluorescence microscopy and fluorescein-conjugated Pisum sativum agglutinin (“FITC-PSA”) staining, the staining patterns of which are known to indicate the status of the acrosomal membrane.

To validate the general experimental protocol, the motility of whole semen treated with N-9 for 5 min was determined using DIC microscopy and PI staining Results are presented in Table 1.

TABLE 1

| Treatment | % Motility | % Death |
|---|---|---|
| (none) | 62% | 28% |
| 0.001% N-9 | 43% | 38% |
| 0.005% N-9 | 60% | 60% |
| 0.01% N-9 | <1% | 67% |
| 0.1% N-9 | 0% | 94% |

The effects of F5-TEG on sperm motility and viability are presented in Table 2.

TABLE 2

| Treatment | % Motility | % Death |
|---|---|---|
| (none) | 62% | 28% |
| 4% F5-TEG (5 min) | 0% | 100% |
| 5% F5-TEG (5 min) | 0% | 96% |
| 6% F5-TEG (5 min) | 0% | 100% |
| 7% F5-TEG (5 min) | 0% | 98% |
| 8% F5-TEG (5 min) | 0% | 99% |
| 9% F5-TEG (5 min) | 0% | 100% |
| 10% F5-TEG (5 min) | 0% | 100% |
| 10% F5-TEG (10 min) | 0% | 100% |
| 12% F5-TEG (5 min) | 0% | 96% |
| 15% F5-TEG (5 min) | 0% | 94% |
| 15% F5-TEG (10 min) | 0% | 100% |

The effects of F7-TEG on sperm motility and viability are presented in Table 3.

TABLE 3

| Treatment | % Motility | % Death |
|---|---|---|
| (none) | 62% | 28% |
| 5% F7-TEG (5 min) | 0% | 97% |
| 5% F7-TEG (10 min) | N/A | 99% |
| 6% F7-TEG (5 min) | 0% | 98% |
| 7% F7-TEG (5 min) | 0% | 99% |
| 8% F7-TEG (5 min) | 0% | 100% |
| 9% F7-TEG (5 min) | 0% | 100% |
| 10% F7-TEG (5 min) | 1% | 95% |
| 12% F7-TEG (5 min) | 0% | 99% |
| 15% F7-TEG (5 min) | 0% | 98% |
| 15% F7-TEG (10 min) | 0% | 100% |

The effects of F₃CCF₂CH₂O(CH₂)₃SO₃Na (“F5-PS”) on sperm motility and viability are presented in Table 4.

TABLE 4

| Treatment | % Motility | % Death |
|---|---|---|
| (none) | 62% | 28% |
| 6% F5-PS (5 min) | 1% | 91% |
| 6% F5-PS (10 min) | N/A | 99% |
| 9% F5-PS (5 min) | 0% | 98% |
| 9% F5-PS (10 min) | 0% | 96% |
| 12% F5-PS (5 min) | 0% | 99% |

The effects of F₃C(CF₂)₂CH₂O(CH₂)₃SO₃Na (“F7-PS”) on sperm motility and viability are presented in Table 5.

TABLE 5

| Treatment | % Motility | % Death |
|---|---|---|
| (none) | 62% | 28% |
| 4% F7-PS (5 min) | <1% | 90% |
| 4% F7-PS (10 min) | N/A | 99% |
| 5% F7-PS (5 min) | <1% | 96% |
| 5% F7-PS (10 min) | 0% | 96% |
| 7% F7-PS (5 min) | 0% | 100% |

### Example 2

### Acrosome Staining

Staining with fluorescein isothiocyanate Pisium sativum agglutinin (“FITC-PSA”) is known to stain components of the acrosome, and the staining pattern differs depending on the condition of the acrosomal membrane. This technique may be used to identify whether the acrosome is intact. Various patterns of fluorescence are seen after labeling of air-dried human spermatozoa with FITC lectins, including a bright acrosome (Type I), a medium acrosome (Type II), an equatorial segment (Type III), and no labeling (Type IV). See, e.g., Mortimer et al., “Specific Labelling by Peanut Agglutinin of The Outer Acrosomal Membrane of The Human Spermatozoon,” J. Reprod. Fert. 81, 127-35 (1987). Averages of FTIC-PSA results are presented in Table 6, which includes percentages of acrosomes compromised and types of sperm visualized in the control and treated samples.

TABLE 6

| | | | | | Acrosome | Acrosome |
|---|---|---|---|---|---|---|
| | Type | Type | Type | Type | Compro- | Not Compro- |
| Treatment | I | II | III | IV | mised | mised |
| (none) | 58% | 27% | 1% | 14% | 42% | 58% |
| 5% F5-TEG | 5% | 28% | 3% | 64% | 95% | 5% |
| 6% F5-TEG | 2% | 3% | 1% | 93% | 98% | 2% |
| 10% F5-TEG | 33% | 26% | 2% | 38% | 67% | 33% |
| 5% F7-TEG | 13% | 59% | <1% | 28% | 87% | 13% |
| 6% F7-TEG | 13% | 25% | 5% | 58% | 88% | 13% |
| 10% F7-TEG | 4% | 34% | 3% | 59% | 96% | 4% |
| 3% F7-PS | 64% | 13% | 0% | 22% | 36% | 64% |
| 4% F7-PS | 0% | 4% | <1% | 95% | 100% | 0% |
| 7% F7-PS | 59% | 40% | 0% | 1% | 41% | 59% |

The natural acrosome reaction yields predominantly (Type III) sperm stained with a bright equatorial band. Both the soluble content of the acrosome compartment and the residual exposed outer acrosomal membrane are thought to be important for natural fertilization. By contrast, sperm treated with fluorous surfactants show predominantly the Type IV pattern, indicating that the treatment has bypassed the natural acrosome reaction and stripped these cells of acrosomal components, thereby rendering them incapable of fertilization.

### Example 3

### Effects of Fluorous Surfactants on HeLa (Cervix) Cells

The effects of nonoxynol-9 and various fluorous surfactants on HeLa cells, which are a laboratory model for human cervical cells, were studied. While N-9 was observed to kill essentially all sperm cells in 5 min at 1.76 mM, an equivalent exposure to HeLa cells for 30 min was observed by microscopy to produce significant morphological changes. In contrast, the same morphological changes were either absent or significantly attenuated when HeLa cells were exposed to a spermicidally effective concentration of a fluorous surfactant. Accordingly, it is believed that use of a spermicidal fluorous surfactant will be less irritating to human vaginal tissue.

### Example 4

### Effects of Fluorous Surfactants on Microbes

Fluorous surfactants also disrupt the membranes of pathogenic microbes, and they therefore have microbicidal activities. Preferably, they are toxic to pathogenic microorganisms, yet compatible with the flora normally found in human mucosal (e.g., vaginal) tissues. The effects of various fluorous surfactants on some laboratory models of pathogenic microorganisms (Escherichia coli, Saccharomyces cerevisiae, and Candida albicans) were studied.

For E. coli, conditions examined resulted in greater inhibition than was seen for comparable exposure to nonoxynol-9. For example, treatment with F7-TEG at final concentrations of 1% or greater for periods of 3 hours or longer rendered E. coli unable to grow. F5-TEG showed less inhibition than comparable doses of F7-TEG, and both of these compounds caused greater inhibition to growth of E. coli than their non-fluorous homologs. A comparable inhibition was produced by exposure of E. coli to sodium 3-(2,2,3,3,4,4,4-heptafluoro-butoxy)-propane-1-sulfonate (“F7-PS”) at 250 mM.

For the fungal species, none of the fluorous surfactant treatments tested resulted in inhibition of growth of Saccharomyces cerevisiae. By contrast, inhibition was observed for treatments of Candida albicans. For example, exposure for 3 hours or longer to 5% F7-TEG gave substantial inhibition of Candida. F5-TEG treatment was somewhat less inhibitory to Candida than was F7-TEG. At concentrations of 400 mM F7PS, exposure times in the order of 24 hours gave significant inhibition of growth of Candida, but exposures limited to 3 or 6 hours allowed recovery of growth.

### Example 5

### Synthesis of Non-Ionic Fluorous Surfactants

Various fluorinated alcohols, e.g., F₃C(CF₂)₁₋₅CH₂OH, were reacted in an organic solvent in the presence of a base with tosyl chloride to produce corresponding fluorous tosylates, which were isolated, purified, and characterized, e.g., by GC/MS and NMR. The resulting fluorous tosylates were then reacted in an organic solvent in the presence of a base with a polyethylene glycol, e.g., H(OCH₂CH₂)₃₋₄OH, to produce the corresponding non-ionic fluorous surfactants, which were isolated by chromatography and characterized by NMR. In this matter, the following compounds were prepared:

F5-triethylene glycol (“F5-TEG”), F₃CCF₂CH₂(OCH₂CH₂)₃OH

F7-triethylene glycol (“F7-TEG”), F₃C(CF₂)₂CH₂(OCH₂CH₂)₃OH

F5-tetraethylene glycol, F₃CCF₂CH₂(OCH₂CH₂)₄OH

F7-tetraethylene glycol, F₃C(CF₂)₂CH₂(OCH₂CH₂)₄OH

F13-tetraethylene glycol, F₃C(CF₂)₅CH₂(OCH₂CH₂)₄OH

Using a protecting group strategy, i.e., protection of one hydroxyl group of the polyethylene glycol as an ether using 3,4-dihydro-2H-pyran (“DHP”), increased reaction yields.

### Example 6

### Synthesis of Anionic Fluorous Surfactants

Various fluorinated alcohols, e.g., F₃C(CF₂)₅₋₇CH₂OH, were reacted in an organic solvent in the presence of a base with propane sultone or butane sultone to produce corresponding fluorous propane-1-sulfonates, which were isolated, purified (e.g., by recrystallization from methanol), and characterized (e.g., by NMR). In this matter, the following compounds were prepared:

“F13-PS,” F₃C(CF₂)₅CH₂O(CH₂)₃SO₃Na

“F17-PS,” F₃C(CF₂)₇CH₂O(CH₂)₃SO₃Na

“F13-BS,” F₃C(CF₂)₅CH₂O(CH₂)₄SO₃Na

“F17-BS,” F₃C(CF₂)₇CH₂O(CH₂)₄SO₃Na

While this description is made with reference to exemplary embodiments, it will be understood by those skilled in the art that various changes may be made and equivalents may be substituted for elements thereof without departing from the scope. In addition, many modifications may be made to adapt a particular situation or material to the teachings hereof without departing from the essential scope. Also, there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended Claims not be limited to the particular embodiment disclosed herein.

1. A method of altering at least one membrane of a spermatozoon comprising a step of contacting at least one spermatozoon with one or more fluorous surfactants.

2. The method according to claim 1, wherein the one or more fluorous surfactants each comprise at least one fluorine atom, have a molecular weight under about 500, and are at least partially soluble in water.

3. The method according to claim 1, wherein the one or more fluorous surfactants are selected from the group consisting of non-ionic fluorous surfactants, anionic fluorous surfactants, cationic fluorous surfactants, and combinations thereof.

4. The method according to claim 3, wherein the one or more fluorous surfactants are selected from the group consisting of non-ionic fluorous surfactants.

5. The method according to claim 4, wherein the non-ionic fluorous surfactants each comprise a fluorine-substituted lower alkyl group covalently bonded to a polyoxyalkyl group.

6. The method according to claim 5, wherein the non-ionic fluorous surfactants are selected from the group of compounds having the following chemical structure:
F₃C[CF₂]ₙCH₂O[CH₂CH₂O]ₘH
wherein n is an integer from 1 to 10, and m is an integer from 1 to 10.

7. The method according to claim 6, wherein n is an integer from 1 to 5.

8. The method according to claim 6, wherein m is an integer from 3 to 6.

9. The method according to claim 3, wherein the one or more fluorous surfactants are selected from the group consisting of anionic fluorous surfactants.

10. The method according to claim 9, wherein the anionic fluorous surfactants each comprise a fluorine-substituted lower alkyl group covalently bonded via an oxygen atom to a lower alkyl sulfonic acid.

11. The method according to claim 10, wherein the anionic fluorous surfactants are selected from the group of compounds having the following chemical structure:
F₃C[CF₂]ₚO[CH₂]_{q}SO₃⁻X⁺
wherein p is an integer from 1 to 10, q is an integer from 1 to 10, and X⁺ is a physiologically acceptable cation.

12. The method according to claim 11, wherein p is an integer from 1 to 7.

13. The method according to claim 11, wherein q is an integer from 3 to 6.

14. The method according to claim 11, wherein X⁺ is a sodium, lithium, potassium, or tetraalkyl ammonium ion.

15. The method according to claim 3, wherein the one or more fluorous surfactants are selected from the group consisting of cationic fluorous surfactants.

16. The method according to claim 15, wherein the cationic fluorous surfactants each comprise a fluorine-substituted lower alkyl group covalently bonded to a lower alkyl ammonium ion.

17. The method according to claim 16, wherein the cationic fluorous surfactants are selected from the group of compounds having the following chemical structure:
F₃C[CF₂]ᵥ[CH₂]_{w}NR₃⁺Y⁻
wherein v is an integer from 1 to 10, w is an integer from 1 to 10, each R is independently a hydrogen or lower alkyl group, and Y⁻ is a physiologically acceptable anion.

18. The method according to claim 17, wherein v is an integer from 1 to 7.

19. The method according to claim 17, wherein w is an integer from 3 to 6.

20. The method according to claim 17, wherein each R is a hydrogen atom.

21. The method according to claim 17, wherein Y⁻ is a chloride or bromide ion.

22. The method according to claim 1, wherein the spermatozoon is a human spermatozoon.

23. The method of claim 1, wherein the step of altering prevents fertilization of at least one ovum or alters the motility of viability of a spermatozoon.

24. A spermicidal composition comprising one or more fluorous surfactants.

25. The spermicidal composition according to claim 24, further comprising one or more additional agents selected from the group consisting of emulsifiers, gelling agents, stabilizers, antioxidants, osmolality adjusting agents, buffers, isotonic agents, water, glycerol, propylene glycol, liquid silicone, liquid polyethylene glycol, lipids, perfumes, flavoring agents, dyes, preservatives, and combinations thereof.

26. The spermicidal composition according to claim 24, wherein the spermicidal composition is in the form of a latex-compatible liquid solution, suspension, or dispersion.

27. A method of preventing infection comprising a step of contacting at least one infectious pathogenic microorganism with one or more fluorous surfactants.

28. The method according to claim 27, wherein the at least one infectious pathogenic microorganism is selected from the group consisting of Neisseria gonorrhoeae, Chlamydia trachomatis, Staphylococcus aureus, Treponema pallidum, Trichomonas vaginalis, Candida albicans, Trichophyton rubrum, Escherichia coli, hepatitis virus, cytomegalovirus, herpes simplex virus, human immunodeficiency virus, human papillomavirus, molluscum contagiosum virus, and subtypes and subspecies thereof.
